(19) United States
(12) Patent Application Publication
(10) Pub. No.: US 2024/0302318 A1
(43) Pub. Date: Sep. 12, 2024

(54) ELECTROCHEMICAL APTAMER SENSORS WITH APTAMERS BOUND ADJACENT TO THE ELECTRODE

(71) Applicant: University Of Cincinnati, Cincinnati, OH (US)

(72) Inventor: Jason Charles Heikenfeld, Cincinnati, OH (US)

(21) Appl. No.: 18/255,896

(22) PCT Filed: Sep. 24, 2021
(86) PCT No.: PCT/US2021/051960

§ 371 Date: Jun. 5, 2023

Related U.S. Application Data
(60) Provisional application No. 63/150,975, filed on Feb. 18, 2021.
(60) Provisional application No. 63/122,003, filed on Dec. 7, 2020.

(51) Int. Cl. G01N 27/327; B01L 3/00; G01N 33/543

(52) U.S. Cl. CPC G01N 27/3276; B01L 3/502761; G01N 27/3277; G01N 33/5438; B01L 2200/0647; B01L 2200/16; B01L 2300/0645

(57) ABSTRACT

An aptamer sensing device is provided. The aptamer sensing device 200 includes at least one electrode 220. The aptamer sensing device further includes at least one binding feature 230. The aptamer sensing device further includes a plurality of aptamers 270 attached to the binding feature and not individually attached to the electrode, the aptamers further having an attached redox couple 272. The aptamer sensing device is configured to accept a sample fluid 242 including at least one analyte, and the binding feature is positioned relative to the electrode such that a binding of the analyte to the aptamers causes a shape change configuration in the binding feature that increases or decreases a charge transfer from the redox couple to the electrode.

### CROSS-REFERENCE TO RELATED APPLICATIONS

This application claims priority to, and the benefit of the filing date of, U.S. Provisional Application No. 63/122,003 filed Dec. 7, 2020 and U.S. Provisional Application No. 63/150,975 filed Feb. 18, 2021, the disclosures of which are incorporated by reference herein in their entireties.

### TECHNICAL FIELD

The present invention relates to the use of electrochemical, aptamer-based (EAB) sensors.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Continuous glucose monitoring for diabetes is a historical achievement in modern diagnostics, and is typically accomplished via the use of enzymatic sensors. Unfortunately, continuous glucose monitoring is an isolated success despite numerous acute needs across the broader field of human disease management. Enabling other continuous biosensors requires a generalizable biosensor platform beyond enzymatic sensors, because enzymatic sensors are limited to high-concentration metabolites (e.g., glucose, lactate, ethanol, etc.). With the advent of electrochemical aptamer-based sensors, this much-needed generalizable platform arrived, and has resulted in dozens of compelling in-vivo aptamer-based sensor demonstrations in animals. EAB sensor development now even includes calibration-free operation and powerful sensor-drift correction methods.

With the initial invention and demonstration of aptamer-based sensors now nearly two decades old, a reasonable question, then, is why there is no clinical adoption of an aptamer-based sensor. In part, this is because device longevity and sensitivity remain a significant challenge. Conventional aptamer-based sensors rely on a redox-tagged aptamer that is chemically bound to the electrode it is measured on. This electrode-bound-aptamer architecture imparts a penalty by attaching the aptamer monolayer to an electrochemically-active electrode which is an intrinsically unstable configuration. Clearly a significant benefit could be realized if a radical new EAB design can be developed that resolves this challenge. Such a breakthrough would then accelerate the clinical proliferation of biosensors beyond just diabetes and glucose.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

Many of the drawbacks and limitations stated above can be resolved by creating novel and advanced interplays of chemicals, materials, sensors, electronics, microfluidics, algorithms, computing, software, systems, and other features or designs, in a manner that affordably, effectively, conveniently, intelligently, or reliably brings sensing technology into proximity with sample fluids containing at least one analyte of interest to be measured.

Embodiments of the disclosed invention are directed to electrochemical aptamer-based sensors where the aptamer is physically bound adjacent to the electrode without being directly bound to the electrode itself.

And so, one aspect of the present invention is directed to and aptamer-based device. The device includes at least one electrode, and at least one binding feature. The device further includes a plurality of aptamers attached to the binding feature and not individually attached to the electrode, the aptamers further having an attached redox couple. The device is configured to accept a sample fluid including at least one analyte, and the binding feature is positioned relative to the electrode such that a binding of the analyte to the aptamers causes a shape change configuration in the binding feature that increases or decreases a charge transfer from the redox couple to the electrode.

### BRIEF DESCRIPTION OF THE DRAWINGS

The objects and advantages of the disclosed invention will be further appreciated in light of the following detailed descriptions and drawings in which:

FIG. 1 is a cross-sectional view of a device according to a conventional aptamer sensor device.

FIG. 2 is a cross-sectional view of a device according to an embodiment of the disclosed invention.

FIG. 3 is a cross-sectional view of a device according to an embodiment of the disclosed invention.

### DEFINITIONS

As used herein, the term “about,” when referring to a value or to an amount of mass, weight, time, volume, pH, size, concentration or percentage is meant to encompass variations of ±20% in some embodiments, ±10% in some embodiments, ±5% in some embodiments, ±1% in some embodiments, ±0.5% in some embodiments, and ±0.1% in some embodiments from the specified amount, as such variations are appropriate to perform the disclosed method.

As used herein, the term “aptamer” means a molecule that undergoes a conformation change as an analyte binds to the molecule, and which satisfies the general operating principles of the sensing method as described herein. Such molecules are, e.g., natural or modified DNA, RNA, or XNA oligonucleotide sequences, spiegelmers, peptide aptamers, and affimers. Modifications may include substituting unnatural nucleic acid bases for natural bases within the aptamer sequence, replacing natural sequences with unnatural sequences, or other suitable modifications that improve sensor function.

As used herein, the term “aptamer sensor” means at least one sensor that uses redox-reporter tagging of an aptamer, and a change electrical signal as a result of a shape conformation change of the aptamer as the aptamer binds with a target analyte.

As used herein, the term “aptamer” means a molecule that undergoes a conformation change as an analyte binds to the molecule, and which satisfies the general operating principles of the sensing method as described herein. Such molecules are, e.g., natural or modified DNA, RNA, or XNA oligonucleotide sequences, spiegelmers, peptide aptamers, and affimers. Modifications may include substituting unnatural nucleic acid bases for natural bases within the aptamer sequence, replacing natural sequences with unnatural sequences, or other suitable modifications that improve sensor function.

The devices and methods described herein encompass the use of sensors. A sensor, as used herein, is a device that is capable of measuring the concentration of a target analyte in a sample fluid. As used herein, an “analyte” may be any inorganic or organic molecule, for example: a small molecule drug, a metabolite, a hormone, a peptide, a protein, a carbohydrate, a nucleic acid, a chemical, a particle, or any other composition of matter. The target analyte may comprise a drug. The drug may be of any type, for example, including drugs for the treatment of cardiac system, the treatment of the central nervous system, that modulate the immune system, that modulate the endocrine system, an antibiotic agent, a chemotherapeutic drug, or an illicit drug. The target analyte may comprise a naturally-occurring factor, for example a hormone, metabolite, growth factor, neurotransmitter, etc. The target analyte may comprise any other species of interest, for example, species such as pathogens (including pathogen induced or derived factors), nutrients, and pollutants, etc.

### DETAILED DESCRIPTION OF THE INVENTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Certain embodiments of the disclosed invention show sensors as simple individual elements. It is understood that many sensors require two or more electrodes, reference electrodes, or additional supporting technology or features which are not captured in the description herein. Sensors measure a characteristic of an analyte. Sensors are preferably electrical in nature, but may also include optical, chemical, mechanical, or other known biosensing mechanisms. Sensors can be in duplicate, triplicate, or more, to provide improved data and readings. Sensors may provide continuous or discrete data and/or readings. Certain embodiments of the disclosed invention show sub-components of what would be sensing devices with more sub-components needed for use of the device in various applications, which are known (e.g., a battery, antenna, adhesive), and for purposes of brevity and focus on inventive aspects, such components may not be explicitly shown in the diagrams or described in the embodiments of the disclosed invention. All ranges of parameters disclosed herein include the endpoints of the ranges.

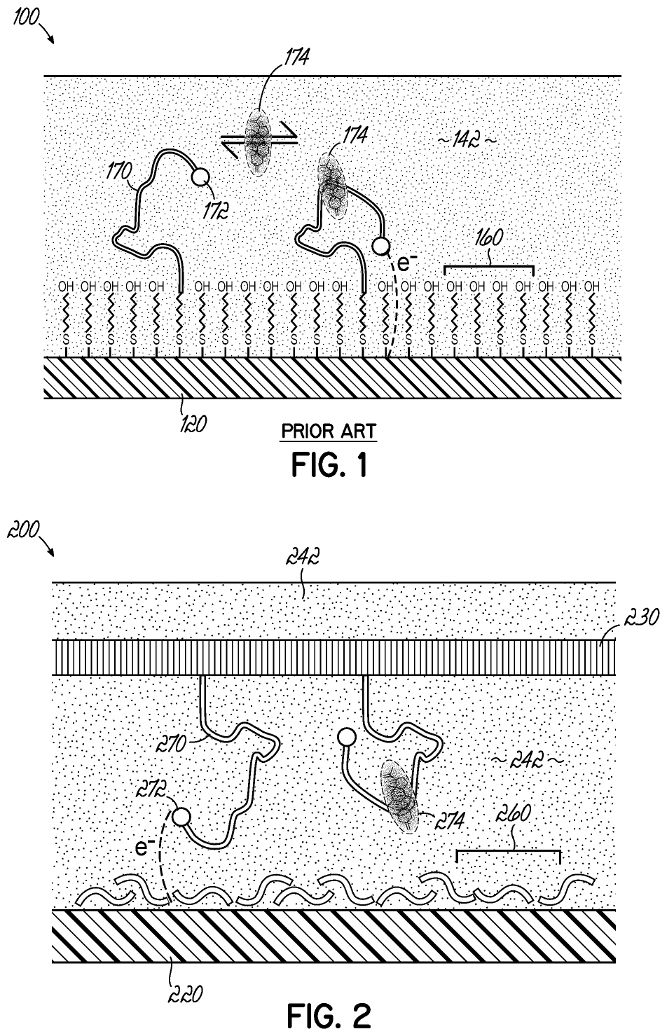

With reference to FIG. 1, in a conventional aptamer sensor, a device 100 includes an electrode 120, passivated by at least one molecule such as mercaptohexanol 160, and functionalized with an electrode-bound aptamer 170 with an attached redox couple 172. When placed in a solution such as a sample fluid 142 with an analyte 174, the analyte 174 binds to the aptamer 170, and the binding of the analyte 174 to the aptamer 170 causes a shape conformation change that further results in an increase (as shown) or decrease (not shown) in charge transfer to the electrode 120 via the redox couple 172. This conventional device is taught in order to illustrate performance limitations that are resolved by the present invention. The electrode-bound aptamer architecture attaches the aptamer monolayer to an electrochemically-active electrode which is an intrinsically unstable configuration. Both the aptamer-electrode and passivating molecule-electrode bond are known to degrade over time periods as short as several hours.

With reference to FIG. 2 where like numerals refer to like features, in an embodiment of the present invention, a device 200 includes an electrode 220, having a passivating layer 260 associated therewith. In some embodiments, the passivating layer 260 is an unnatural material such as mercaptohexanol. In other embodiments, the passivating layer 260 is one or more natural solutes (amino acids, polypeptides, etc.) from the sample fluid 242, such as cysteine groups on solutes that may bond to a gold surface or hydrophobic groups on solutes that may have affinity for a hydrophobic surface such as carbon. The device 200 also includes a binding feature 230, to which aptamers 270 are chemically bound. In one embodiment, the binding feature 230 is a substrate. In another embodiment, the binding feature 230 is a membrane. In yet another embodiment, the binding feature is a nanoparticle. As a result, the aptamers 270 do not need to be, and indeed may not be, individually bound to the electrode 220 and, therefore, are not subject to bonding degradation due to that electrode 220. In addition, with natural solutes in the sample fluid 242 at least partially passivating the electrode 220, degradation or change in the passivating layer 260 is less of a concern because the passivating layer 260 naturally repopulates with new solutes over time as older solutes are de-bonded from electrode 220 or degraded. When placed in a solution such as a sample fluid 242 with an analyte 274, the analyte 274 binds to the aptamer 270, and the binding of the analyte 274 to the aptamer 170 causes a shape conformation change that further results in an increase (not shown) or decrease (as shown) in charge transfer to the electrode 220 via the redox couple 272.

With further reference to FIG. 2, the binding feature 230 could be a planar substrate so long as there is at least one route for solutes in the sample fluid 242 to reach the aptamers 270. In one embodiment, the binding feature 230 is glass or silicon. In another embodiment, the binding feature 230 may also serve as a membrane to allow rapid analyte diffusion from sample fluid 242 to the aptamer 270, with membrane molecular weight cutoffs ranging from 100s to 10,000s of Da's or more, to keep large molecules, cells, and other foulants outside of the device that could rapidly degrade the device (thick fouling layer formation, proteases that attack the aptamer, etc.). Non-limiting embodiments of membrane materials include porous glass where the aptamer 270 is bound to the glass with silane groups, porous carbon where the aptamer 270 is bound with an amine group or click-chemistry, or cellulose acetate, gold coated membranes, dialysis, nano-filtration, ultra-filtration, or other suitable membranes and attachment chemistries.

With further reference to FIG. 2, the aptamers 270 and redox couples 272 may be in proximity with the electrode 220. The precise proximity between electrode 220 and binding feature 230 will depend on aptamer 270 design (length nm's to 10 nm or more, folding etc.). There are two general strategies for providing this proximity: soft binding features 230 (those that would be mechanically compliant), and rigid binding features 230 (those that would not be mechanically compliant). Non-limiting embodiments of soft binding features include conventional acetate, polymethylesiloxane, and other types of filtration membranes, which typically have a very smooth and pliable interface on at least one side. These membranes may be used with or without a spacer layer such as nanoparticles, proteins such as albumin (several nm's or more), or other suitable spacers and pressed against the electrode 220. Rigid binding features 230 can be rough (>5 nm rms roughness) or smooth (<5 nm rms roughness). For example, rigid and porous glass or porous silicon can be made smooth by forming them onto a smooth template, polishing, or other suitable means. This allows spacers such as mono-disperse polymer or glass nanoparticles, or other suitable spacer techniques in the semiconductor industry or other industries, to provide an average spacing. Spacers between binding feature and electrode may also include a particle. Examples of particles that may be used as a spacer include a nanoparticle, a large molecule, and double stranded DNA.

In one embodiment, any of the binding features 230, including membranes or other materials, are held by pressure to be adjacent to the electrode, and the binding feature 230 may be positioned relative to the electrode 220 by mechanical pressure. Non-limiting embodiments of this approach include a plastic or stainless steel mesh or plate or for example a sponge pressed against binding feature 230 (not shown). In another embodiment, any of the binding features 230 may be held adjacent to the electrode 220 using physical attachment, and the binding feature 230 is positioned relative to the electrode 220 by physical bonding or chemical bonding. Non-limiting embodiments of this approach include epoxies, anodic bonding, adhesives, chemical bonding or bridging, or other suitable methods. Generally, the average distance of the binding feature 230 should be far enough to allow free mobility (binding, unbinding) for the aptamer 270, yet close enough such that the redox couple 272, such as methylene blue, may transfer charge to the electrode 220. For binding features 230 that are not atomically smooth, one or more pockets >5 nm in depth may exist that allow the aptamer 270 to have adequate mobility and the binding feature 230 can then be pressed into physical contact with the electrode 220. Alternately, the binding feature 230 and/or the electrode 220 may be rough and contain pockets >5 nm in depth and pressed against each other, for example with a carbon paste electrode that is pressed semi-smooth or semi-polished. In an embodiment, the binding feature 230 includes a pocket facing the electrode, and the pocket has an average depth of >5 nm. In another embodiment, the electrode 220 includes a pocket facing the binding feature 220, and the pocket has an average depth of >5 nm. In an embodiment, the binding feature 230 is positioned relative to the electrode 220 at a distance selected from the group consisting of <100 nm, <20 nm, <10 nm, and <5 nm.

In some embodiments, the binding feature 230 surface is within 100 nm, and in other embodiments within 20 nm, 10 nm, or even 5 nm of the surface of the electrode 220. In some embodiments, the density of aptamers 270 bonded to the binding feature 230 and adjacent to the electrode 220 is 1E9 to 1E13 per cm². In other embodiments, the density of aptamers 270 bonded to the binding feature 230 and adjacent to the electrode 220 is 1E10 to 1E12 per cm² of working electrode area to allow a strong electrochemical while not restraining free movement of the aptamers 270.

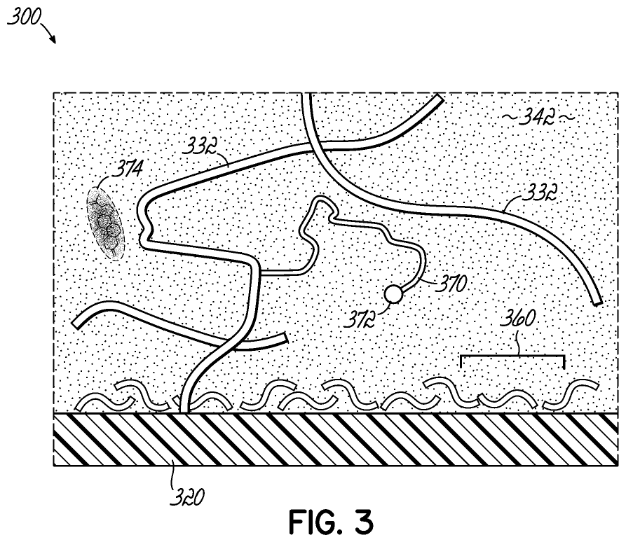

With reference to FIG. 3 where like numerals refer to like features, in an embodiment of the present invention, a device 300 comprises a binding matter is used as the binding feature 330 instead of a binding substrate as the binding feature, as shown in FIG. 2. The binding feature 332 may be any material that brings aptamers 370 adjacent to the electrode 320 without their function being directly impacted by being individually chemically bound to the electrode 320. In some embodiments, the binding feature 332 is pressed against the electrode 320 or is physically or chemically bound to one location or a plurality of locations on the electrode 320. In one embodiment, the binding feature 332 is a hydrogel. In another embodiment, the binding feature 332 is an acrylamide hydrogel that is 100 nm thick and the aptamer 370 bound to the hydrogel using acrydite attachment chemistry. Even in the case where the binding feature 332 is bound to the electrode 320, it overcomes previous limitations described for FIG. 1 because gradual loss of binding between binding feature 332 and electrode 320 will negatively impact device performance so long as enough binding is retained such that binding feature 332 and electrode 320 do not delaminate from each other. As a result, binding feature 332 may only need to retain, in different embodiments, 90%, 50%, 10%, 1% or 0.2% of its initial bonds to the electrode 320. Precise distancing of aptamers 370 to electrode 320 may not always be required, as analyte 374 binding can also change aspects not just in terms of magnitude of redox current between redox tag 370 and electrode 320 but also altered peak frequency response with square wave voltammetry and other factors that change as the aptamer 370 binds to the analyte 374.

With reference to embodiments of the present invention, binding features or matter used as a binding feature can also be micro or nanoparticles. For example, round or flat-edged magnetic nanoparticles that are held near the substrate via a magnet behind electrode 220, 320 (not shown). Nanoparticles or other suitable features or matters that have attached aptamer can also rely on precise spacers, such as a DNA double helix that is bonded to the features or matter with a precise distance such as 5 or 10 or 20 nm. For example, nanoparticles carrying aptamers could allow the electrode 220, 320 to have adjacent to it aptamers at densities of 1E10 to 1E11 or greater per cm² allowing adequate spacing of the aptamers so they do not interfere with each other's switching behaviors.

With reference to an embodiment of the present invention, redox reports such as methylene blue are inherently stable as are aptamers as well (stored in water for >12 months). As a result, the present invention enables a sensing device with a working lifetime, in different embodiments, of at least 1 week, 1 month, 3 months, 12 months. Aptamers can be readily bound using silane or other chemistries to meet such durations of operation. Such devices could also be stored dry in sugar or other preservative materials.

Although not described in detail herein, other steps which are readily interpreted from or incorporated along with the disclosed embodiments shall be included as part of the invention. The embodiments that have been described herein provide specific examples to portray inventive elements, but will not necessarily cover all possible embodiments commonly known to those skilled in the art.

What is claimed is:

1. An aptamer sensing device comprising:
at least one electrode;
at least one binding feature; and
a plurality of aptamers attached to the binding feature and not individually attached to the electrode, the aptamers further having an attached redox couple;
wherein the device is configured to accept a sample fluid including at least one analyte, and the binding feature is positioned relative to the electrode such that a binding of the analyte to the aptamers causes a shape change configuration in the binding feature that increases or decreases a charge transfer from the redox couple to the electrode.

2. The device of claim 1, further comprising a passivating layer on the electrode.

3. The device of claim 2, wherein the passivating layer includes at least one chemical not included in the sample fluid.

4. The device of claim 2, wherein the passivating layer includes at least one chemical included in the sample fluid.

5. The device of claim 1, further comprising at least one pathway configured to allow the analyte to diffuse between the sample fluid and the aptamers.

6. The device of claim 1, wherein the binding feature is a substrate.

7. The device of claim 1, wherein the binding feature is a membrane.

8. The device of claim 1, wherein the binding feature is rigid and not mechanically compliant with the electrode.

9. The device of claim 1, wherein the binding feature is soft and mechanically compliant with the electrode.

10. The device of claim 1, wherein the binding feature has a rms roughness >5 nm.

11. The device of claim 1, wherein the binding feature has a rms roughness <5 nm.

12. The device of claim 1, wherein the binding feature is positioned relative to the electrode by mechanical pressure.

13. The device of claim 1, wherein the binding feature is positioned relative to the electrode by physical bonding.

14. The device of claim 1, wherein the binding feature is positioned relative to the electrode by chemical bonding.

15. The device of claim 1, wherein said binding feature includes a pocket facing the electrode, said pocket having an average depth of >5 nm.

16. The device of claim 1, wherein said electrode includes a pocket facing the binding feature, said pocket having an average depth of >5 nm.

17. The device of claim 1, wherein the binding feature is positioned relative to the electrode at a distance selected from the group consisting of <100 nm, <20 nm, <10 nm, and <5 nm.

18. The device of claim 1, wherein the aptamers bound to the binding feature have a surface density between 1E10 and 1E12 per cm² of electrode area.

19. The device of claim 1, wherein the aptamers bound to the binding feature have a surface density between 1E9 and 1E13 per cm² of electrode area.

20. The device of claim 1, wherein the binding feature is a hydrogel.

21. The device of claim 20, wherein the device has a lifetime of use, and during that use the hydrogel retains a percentage of its bonds to the electrode selected from the group consisting of >90%, >50%, >10%, >1%, and >0.2%.

22. The device of claim 1, wherein the binding feature is a nanoparticle.

23. The device of claim 1, further comprising at least one spacer between the binding feature and the electrode.

24. The device of claim 23, wherein the spacer is a particle that is at least one of a nanoparticle, a large molecule, and double stranded DNA.

25. The device of claim 1, wherein the device has a lifetime of use, and the lifetime of use is selected from the group consisting of 1 week, 1 month, 3 months, and 12 months.
